(19) United States
(12) Patent Application Publication
(10) Pub. No.: US 2022/0204639 A1
(43) Pub. Date: Jun. 30, 2022

(54) Novel methods for the in vitro processing of cancer cells from one individual to accurately preserve the antigenic architecture of multiple surface abnormalities specific to the individual cancer and for rapidly selecting and amplifying anti-cancer molecules highly specific for cancer stem cells and other abnormalities regardless of their rarity while minimizing collateral damage to normal tissue associated with less specific therapies

(71) Applicant: Bernard Friedenson, Des Plaines, IL (US)

(72) Inventor: Bernard Friedenson, Des Plaines, IL (US)

(21) Appl. No.: 17/139,787

(22) Filed: Dec. 31, 2020

(51) Int. Cl. C07K 16/30; A61P 35/00; C12N 5/09; A01N 1/02

(52) U.S. Cl. CPC C07K 16/30; A61P 35/00; A61K 2039/55; A01N 1/0278; C12N 5/0693

(57) ABSTRACT

This disclosure is in the field of cancer immunotherapy and relates to all cancer types, including but not limited to cancers of the breast, lung, prostate, pancreas, colon, bladder, brain, head-neck, kidney, esophagus, skin, and blood cells. The embodiments provide methods for selecting and amplifying specific targeting molecules to use in therapy and diagnostic testing. Targets include but are not necessarily limited to architecturally preserved, usually heterogeneous specific surface structures present on individual cancer cells and cancer stem cells but not on non-malignant cells. The embodiments are novel in that they provide these binding molecules for one individual's cancer regardless of the rarity of an individual cancer cell or stem cell and promptly enough to initiate treatment without requiring lengthy immunization or hybridoma production that are current art.

### REFERENCES

U.S. Patent Documents

| | | |
|---|---|---|
| 6,794,494 | Sep. 21, 2004 | Young, et al |
| 7,175,846 | Feb. 13, 2007 | Young, et al |
| 7,183,384 | Feb. 27, 2018 | Sun, et al |
| 7,186,808 | Mar. 6, 2007 | Young, et al |
| 7,256,272 | Aug. 14, 2007 | Young, et al |
| 7,314,622 | Jan. 1, 2008 | Arlen, et al |
| 7,355,008 | Apr. 8, 2008 | Stavenhagen, et al |
| 7,399,835 | Jul. 15, 2008 | Young, et al |
| 7,420,039 | Sep. 2, 2008 | Young, et al |
| 7,488,475 | Feb. 10, 2009 | Young, et al |
| 7,560,095 | Jul. 14, 2009 | Sun, et al |
| 7,671,010 | Mar. 2, 2010 | Arap, et al |
| 8,323,902 | Dec. 4, 2012 | Ernst, et al |
| 8,343,497 | Jan. 1, 2013 | Shi, et al |
| 8,372,954 | Feb. 12, 2013 | Tanha, et al |
| 8,440,188 | May 14, 2013 | Multhoff, T |
| 8,455,615 | Jun. 4, 2013 | Sanda, et al |
| 8,603,474 | Dec. 10, 2013 | Ritter, et al |
| 8,614,169 | Dec. 24, 2013 | Tainsky, et al |
| 8,697,396 | Apr. 15, 2014 | Dall'Acqua, et al |
| 8,802,093 | Aug. 12, 2014 | Johnson, et al |
| 8,802,826 | Aug. 12, 2014 | Tremblay, et al |
| 9,222,938 | Dec. 29, 2015 | Tainsky, et al |
| 9,284,375 | Mar. 15, 2016 | Johnson, et al |
| 9,310,371 | Apr. 12, 2016 | Metodiev, |
| 9,388,249 | Jul. 12, 2016 | Sugioka, et al |
| 9,708,408 | Jul. 18, 2017 | Stavenhagen, et al |
| 9,822,180 | Nov. 21, 2017 | Cobbold, et al |
| 9,884,893 | Feb. 6, 2018 | Glanville |
| 9,889,197 | Feb. 13, 2018 | Johnson, et al |
| 10,055,540 | Aug. 21, 2018 | Yelensky, et al |
| 10,093,738 | Feb. 10, 2016 | Johnson and Huang |
| 10,124,023 | Nov. 13, 2018 | Brentjens et al |
| 10,253,100 | Apr. 9, 2019 | Igawa, et al |
| 10,471,146 | Nov. 12, 2019 | Matsumura, et al |
| 10,538,592 | Jan. 21, 2020 | Lin et al |
| 10,618,965 | Apr. 14, 2019 | Igawa, |
| 10,636,512 | Apr. 28, 2020 | Bloom, et al |
| 10,640,576 | Apr. 1, 2019 | Jang et al |
| 10,709,738 | Jul. 14, 2020 | Schuster et al |

Foreign Patent Documents

| | | |
|---|---|---|
| DE10041342A1 | 2002 Mar. 07 | Zielinski |
| DE10018403 (A1) | 2001 Oct. 31 | Bio Life Science Forschungs & |
| | | Entwicklungsgesellschaft MBH |
| CN108137685 (A) | 2018 Jun. 08 | Vogelstein, et al |

### Other References

Althoff T, Mills, D., et al. Arrangement of electron transport chain components in bovine mitochondrial supercomplex EMBO J. 2011; 30:4652
Anagnostou et al., Evolution of Neoantigen Landscape during Immune Checkpoint Blockade in Non-Small Cell Lung Cancer 2017; Cancer Discovery 7:264-76
Atashzar M, Baharlou R, Karami J., et al. Cancer stem cells: A review from origin to therapeutic Implications. J Cell Physiol. 2020; 235:790-803.
Bailey et al., Comprehensive Characterization of Cancer Driver Genes and Mutations. Cell 2018; 173:371-385
Baik et al. Identification of invadopodia by TKSS staining in human cancer lines and patient tumor samples MethodsX. 2019; 6:718-725
Baselga and Albanell Mechanism of action of anti-HER2 monoclonal antibodies Ann Oncol 2001; 12:S25-41
Bausch-Fluck D, Hofmann A, Bock T, et al. A mass spectrometric-derived cell surface protein atlas. PLoS One. 2015;10(3):e0121314.
Bonnete and Loll, Characterization of New Detergents and Detergent Mimetics by Scattering Techniques for Membrane Protein Crystallization. Methods Mol Biol. 2017; 1635:169-193 Borrega G, Godel J, Ruger P, et al. In the eye of the storm: Immune medated toxicities associated with CAR-T therapy. HemaSphere 2019; 3:e191
Casasent A K, Schalck A, Gao R, et al. Multiclonal Invasion in Breast Tumors Identified by Topographic Single Cell Sequencing. Cell. 2018;172(1-2):205-217 e212.
Chae et al. Maltose-neopentyl glycol (MNG) amphiphiles for solubilization, stabilization and crystallization of membrane proteins. Nat Methods 2010; 7:1003-1008
Charvolin D, Perez J B, Rouviere F, et al. The use of amphipols as universal molecular adapters to immobilize membrane proteins onto solid supports. Proc Natl Acad Sci USA. 2009; 106(2):405-410.
Corradetti B, Taraballi F, Martinez J, et al. Hyaluronic acid coatings as a simple and efficient approach to improve MSC homing toward the site of inflammation. Scientific Reports 2017; 7:7991.
Dahmke, et al. Correlative Fluorescence- and Electron Microscopy of Whole Breast Cancer Cells Reveals Different Distribution of ErbB2 Dependent on Underlying Actin Front Cell Dev Biol; 2020; 6:521
Della Pia E A, Hansen R W, Zoonens M, Martinez K L. Functionalized amphipols: a versatile toolbox suitable for applications of membrane proteins in synthetic biology. J Membr Biol. 2014;247(9-10):815-826.
Ellsworth R E, Blackburn H L, Shriver C D, Soon-Shiong P, Ellsworth D L. Molecular heterogeneity in breast cancer: State of the science and implications for patient care. Semin Cell Dev Biol. 2017; 64:65-72.
Etzkorn M, Zoonens M, Catoire L J, Popot J L, Hiller S. How amphipols embed membrane proteins: global solvent accessibility and interaction with a flexible protein terminus. J Membr Biol. 2014;247(9-10):965-970.
Friedenson B. A Genome Model to Explain Major Features of Neurodevelopmental Disorders in Newborns. Biomed Inform Insights. 2019; 11:1178222619863369.
Friedenson B. Dewey defeats Truman and cancer statistics. J Natl Cancer Inst. 2009; 101(16):1157.
Friedenson B. Mutations in Breast Cancer Exome Sequences Predict Susceptibility to Infections and Converge on the Same Signaling Pathways Journal of Genomes and Exomes Journal of Genomes and Exomes 2015; 4:1-28.
Friedenson B. Mutations in components of antiviral or microbial defense as a basis for breast cancer. Funct Integr Genomics. 2013; 13(4):411-424.
Gai J H, Gong P T, Li J. H., et al. Cell budding from pre-invasive tumors: Intrinsic precursor of invasive breast lesions? Exp Ther Med. 2011; 2(4):633-639.
Henke E. Nandigama R, and Ergun S. Extracellular matrix in the tumor micro-environment and its impact on cancer therapy. Frontiers Mol Biosciences 2019; 6: article 160
Hesketh S J, Klebl D P, Higgins A J, et al. Styrene maleic-acid lipid particles (SMALPs) into detergent or amphipols: An exchange protocol for membrane protein characterisation. Biochim Biophys Acta Biomembr. 2020;1862(5):183192.
Hoogenboom et al., Antibody phage display technology and its applications 1998; Immunotechnology 4:1-20
Hoshino et al. 2013. Signaling inputs to invadopodia and podosomes J. Cell Science 126:2979-2989
Jaafar H, et al. Distinctive Features of Advancing Breast Cancer Cells and Interactions with Surrounding Stroma Observed Under the Scanning Electron Microscope Asian Pacific Journal of Cancer Prevention, 2012; 13: 1305-1310.
Jager et al. High level transient production of recombinant antibodies and antibody fusion proteins in HEK293 cells BMC Biotechnology 2013, 13:52 Jamal-Hanjani, Tracking the Evolution of Non-Small-Cell Lung Cancer2017; NEJM 376:2109-2121
Jiang et al., 2017; Lipids and ions traverse the membrane by the same physical pathway in the nhTMEM16 scramblase. eLife 6:e28671
Keskin D, Ananappa A, Sun J, et al. Neoantigen vaccine generates intratumoral T cell responses in phase Ib glioblastoma trial. Nature 2019; 565:235-239.
Kleinschmidt J H, Popot J L. Folding and stability of integral membrane proteins in amphipols. Arch Biochem Biophys. 2014; 564:327-343.
Llado et al., Regulation of the cancer cell membrane lipid composition by NaCHOleate. Biochim Biophys Acta 2014; 1838:1619.
Le Bon C, Popot J L, Giusti F. Labeling and functionalizing amphipols for biological applications. J Membr Biol. 2014; 247(9-10):797-814.
Ledsgaard L, et al Basics of Antibody Phage Display Technology. Toxins (Basel). 2018;10 Marshall M, Stopforth R., and Cragg M. Therapeutic Antibodies: What Have We Learnt from Targeting CD20 and Where Are We Going? Front. Immunol. 2017; 8:1245
Leitinger B. and Hogg N. Shape and shift changes related to the function of leukocyte integrins LFA-1 and Mac-1. J Leukoc Biol 2001; 69:893-8
Metodieva G, et al. CD74-dependent Deregulation of the Tumor Suppressor Scribble in Human Epithelial and Breast Cancer Cells 2013; Neoplasia. 15(6): 660-668.
Mus-Veteau I. Membrane proteins production for structural analysis. New York: Springer; 2014. Nangalia J and Campbell P J. Genome Sequencing during a Patient's Journey through Cancer. N Engl J Med. 2019; 381(22):2145-2156
Nik-Zainal S, Alexandrov L B, Wedge D C, et al. Mutational processes molding the genomes of 21 breast cancers. Cell. 2012; 149(5):979-993.
Provenzano P P, Eliceiri K W, Campbell J M, et al. Collagen reorganization at the tumor-stromal interface facilitates local invasion. BMC Med 2006; 4, 38.
Qui W, Fi Z, Ku G, et al. Structure and activity of lipid bilayer within a membrane-protein transport. PNAS 2018; 115:12985-12990.
Robinson, J., Halliwell, J. A., McWilliam, H., Lopez, R., Parham, P., and Marsh, S. G. (2013). The IMGT/HLA database. Nucleic Acids Res 41, D1222-1227.
Schumacher and Schreiber, Neoantigens in cancer immunotherapy 2015; Science 348:69-74 Shao et al., 2018 The systeMHC project Nucl Acids Res 46: D1237-1247
Smeal S, Schmitt M, Pereira R, Prasad A and Fisk J. Simulation of the M13 life cycle I: Assembly of a genetically-structured deterministic chemical kinetic simulation. Virology 2017; 500: 259-274.
Stetsenko AaG, A. An Overview of the Top Ten Detergents Used for Membrane Protein Crystallization. Crystals. 2017; 7:197.
Suski J M, Lebiedzinska M, Wojtala A, et al. Isolation of plasma membrane-associated membranes from rat liver. Nat Protoc. 2014; 9(2):312-322.
Tribet C, Diab C, Dahmane T, Zoonens M, Popot J L, Winnik F M. Thermodynamic characterization of the exchange of detergents and amphipols at the surfaces of integral membrane proteins. Langmuir. 2009; 25(21):12623-12634.
Vogelstein and Kinzler, Cancer genes and the pathways they control. Nature Med 2004: 10: 789-799
Wilson, R, Denisin A, Dunn R, Pruitt B, 3D Microwell Platforms for Control of Single Cell 3D Geometry and Intracellular Organization https://ww.bioxiv.org/content/10.1101/2020.07.19.209460v1.full
Yamaguchi. Pathological roles of invadopodia in cancer invasion and metastasis Eur J. Cell Biol. 2012; 91:902-907.
Yoon H, Song J, Ryu C. et al. An efficient strategy for cell-based antibody library selection using an integrated vector system. BMC Biotechnology 2012; 12:62
Zacharakis N, Chinnasamy H, Black M, et al. Immune recognition of somatic mutations leading to complete durable regression in metastatic breast cancer. Nat Med. 2018; 24(6):724-730.
Zhang X, Kim S, Hundal J, et al. Breast Cancer Neoantigens Can Induce CD8(+) T-Cell Responses and Anti-tumor Immunity. Cancer Immunol Res. 2017; 5(7):516-523.
Zoonens M, Giusti F, Zito F, Popot J L. Dynamics of membrane protein/amphipol association studied by Forster resonance energy transfer: implications for in vitro studies of amphipol-stabilized membrane proteins. Biochemistry. 2007; 46(36):10392-10404.
Zoonens M Z, F, Martinez, K. and Popot, J L. Amphipols: A General Introduction and Some Protocols. in I Mus-Veteau (ed) Membrane proteins Production for Structural Analysis. 2014:173-203.

### References to Protein Structures in Figures: Protein Database Accession Number and Publication

2SRC Crystal structures of c-Src reveal features of its autoinhibitory mechanism. Xu, W., Doshi, A., Lei, M., Eck, M. J., Harrison, S. C. (1999) Mol Cell 3: 629-638 Primary Citation of Related Structures: 2SRC
2JCP Banerji, S., Wright, A. J., Noble, M. E. M., et al.Structures of the Cd44-Hyaluronan Complex Provide Insight Into a Fundamental Carbohydrate-Protein Interaction. Nat Struct Mol Biol 2008; 14: 234
7A6E Nosol et al. Cryo-EM structures reveal distinct mechanisms of inhibition of the human multidrug transporter ABCB1. PNAS 2020; 117:26245-53
Primary Citation of Related Structures: 5IPZ, 5JN8, 5JNA, 5JNC, 5JN9, 5KU6 Family Accession PF00194 PDB ID 5JNA Carbonic anhydrase Intrinsic thermodynamics of high affinity inhibitor binding to recombinant human carbonic anhydrase IV. Mickeviciute, A., et al (2018) Eur Biophys J 47: 271-290
4UM8, 4UM9 Crystal structure of alpha V beta 6 DOI: 10.2210/pdb4UM8/pdb Classification: IMMUNE SYSTEM Mutation(s): Yes Deposition Author(s): Dong, X., Springer, T. A. Structural determinants of integrin (3-subunit specificity for latent TGF-β Dong X, Hudson N E, Lu C, Springer T A Nat Struct Mol Biol (2014) 21 p.1091-6
5TCX Crystal structure of human tetraspanin CD81 DOI: 10.2210/pdb5TCX/pdb Classification: CELL INVASION Organism(s): Homo sapiens Expression System: Spodoptera frugiperda Mutation(s): Yes
Deposited: 2016-09-16 Released: 2016-11-09 Deposition Author(s): Zimmerman, B., McMillan, B. J., Seegar, T. C. M., Kruse, A. C., Blacklow, S. C.

### GOVERNMENT INTERESTS

Grant Support

This invention was not supported by a government grant

### DESCRIPTION

### Field of the Invention

The present invention embodiments differ from all prior art by recognizing abnormal cell surface morphology is present in all cancers, and that said abnormal morphology can be targeted by immunotherapy. The immunotherapy is independent of the rarity of the abnormal morphology. The embodiments comprise application to whole cells for processes developed to determine the 3-dimensional structures of pure proteins. The embodiments preserve the conformations of cancer cell surface targets to prepare antibody-based immunotherapy medication specific to treating human cancer in any individual.

### Background of the Invention

DNA mutations in normal cells accumulate throughout life from random errors in DNA copying and individual exposures to mutagens such as radiation or smoking. These processes each leave a characteristic mutation signature on DNA. Comparing this usually benign DNA variation to cancer DNA identifies mutations that drive the cancer process. Mutations in a few cancer “driver” genes give cancer cells a growth or survival advantage. Cancer therapy begins after stratifying disease based on which cancer driver mutations and prognostic markers are present (see Vogelstein and Kinzler, 2004: 10: 789-799; Bailey et al., 2018). This therapeutic strategy assumes that a cancer patient with a driver mutation in gene “X” will benefit from medication specifically for the driver mutation in gene “X”. Unfortunately, this strategy “has always been a bit naïve.” (Nangalia and Campbell, 2019) and has had only limited success. Much prior art has not recognized that many mutations that are not cancer drivers influence how well cancer responds to a driver mutation inhibitor. Because any combination of the six billion bases in the human genome can mutate, cancer has virtually unlimited molecular profiles and mutations. These numbers dwarf the numbers of available drugs. Many cancers do not have driver mutations susceptible to available drugs

Molecular profiles of biopsy samples from the same tumor change at different biopsy sites (Jamal-Hanjani et al. 2017). Cancer stem cells are thought to generate much of this diversity in many types of tumors. These stem cells can self-renew, differentiate to produce diverse tumor cells, and initiate new tumors. Cancer comes back after treatment because available drugs miss cancer stem cells, which typically exist as a small fraction of the tumor (see e.g. Atashzar et al. 2019)

The tumor grows into a complicated, unique mixture that evolves to resist therapy. Many cancers have mutations that inactivate genes, and it is daunting to develop effective medications for cells with missing genes. Many cancer cells contain large DNA deletions, breaks, insertions, or genome rearrangements that available drugs cannot target. Prevalent anti-cancer protocols do nothing to prevent reactions with normal cells. Gene X typically occurs in multiple tissues, so targeting gene X in cancer kills more abundant normal cells.

Current immunotherapy strategies. A significant problem with the prior art of immunotherapy is that it does not admit that cancer cells have many antigens in common with normal cells. Immunotherapy with a monoclonal antibody reagent (e.g., Herceptin) is used for some breast cancers with overexpressed receptors for epidermal growth factor 2 (ERBB2) (Baselga and Albanell, 2001). Many normal cells have epidermal growth factor receptors, so Herceptin causes significant adverse reactions such as brain damage and heart failure. A cell-based approach initially used for blood cancers engineers peripheral blood T-cells to express chimeric antigen receptors. T-cells with the engineered receptor are then returned to the patient to recognize and kill cancer cells. The treatment causes life-threatening toxicities mediated by the immune system (Borrega et al., 2019).

Like Herceptin, virtually all commercial monoclonal antibodies directed against human cancer cells also target normal human cells because they express the same antigen. The presence of cancer antigens on normal cells limits therapeutic effectiveness, increases toxicity, and often requires pausing or stopping therapy. For example, rituximab targets CD20 in lymphoma (Marshal et al., 2017). Healthy B-cells and other organs also express CD20, so adverse reactions include life-threatening infections and heart damage. Similarly, other antibody therapy causes adverse reactions by targeting cancer cell surface molecules shared with normal cells (e.g., Gelber, 7,687,607 Mar. 30, 2010; Gelber, 7,183,389 Feb. 27, 2007; Gelber, 7435554 Oct. 14, 2008; Gelber, 7435415 Oct. 8, 2008). Shared cancer-associated antibody targets are all present on non-malignant cells including mesothelin (Ebel et al., U.S. Pat. No. 8,206,710, Jun. 26, 2012), membrane transporter NaPi2b (SLC34A1) (Ritter et al., U.S. Pat. No. 8,603,474), heat shock protein HSP70 (Multhoff, U.S. Pat. No. 8,440,188, May 14, 2013), TMEFF2 (Bhaskar et al., U.S. Pat. No. 8,257,708, Sep. 4, 2012), oncostatin M receptor (Mather et al. U.S. Pat. No. 8,216,578, Jul. 10, 2012), or a common epitope of p53 (Soloman et al., U.S. Pat. No. 8,207,309, Jun. 26, 2012). Although cell surface forms of interior cell proteins are well-known, treatment can also target an internal organelle protein aberrantly expressed on the cell surface. Sugioka et al. (U.S. Pat. No. 9,388,249 Jul. 12, 2016) prepared cell lines expected to express cell surface antigens representative of all cancers. Sugioka et al. then classified an extensive antibody library according to reactions with cell lines characteristic of one type of cancer. Because of cancer heterogeneity, stromal interactions, and artifacts caused by preparing cell lines, a cell line expressing antigens characteristic of all cancers at any site is impossible in practice.

Another disadvantage of the prior art is that it does not recognize that cancer is associated with damage to the immune system. Our analyses of hundreds of different cancer genomes showed that mutations always include damage to adaptive or innate immunity (see Friedenson, 2013). Moreover, the remnants of human immune responses in cancer reflect diverse human genetic backgrounds and unique histories of exposures to diseases, toxins, and mutagens.

“Neoantigens” or “neoepitopes” are characteristic, distinguishing molecules on cancer cell surfaces. Neoantigens and neoepitopes have been pursued for decades as therapeutic targets, without much success. Neoantigens or neoepitopes should be ideal targets for immunotherapy, but “the vast majority of mutations within expressed genes are not neoantigens that can be recognized by autologous T-cells” (Schumacher and Schreiber, 2015). Cancers of the same type in different individuals do not share neoantigens (Hoogenboom et al., 1998). Many different versions of cancer cell neoantigens are thought to exist, with some at low density in only a few cells. Because the immune response in cancer is damaged, stimulating immunity with a vaccine has been unsuccessful. Patients who received a neoantigen vaccine for glioblastoma generated a T-cell that did not clear the tumor (Keskin et al., 2019).

Purification of cancer cell neoantigens is technically challenging. Currently, it is not generally feasible to select out therapeutic antibodies from comprehensive human antibody libraries. Any single individual cancer neoantigen may be rare, with concentrations so low no antibody in the library is strong enough to find it. High levels of irrelevant antibodies in the library and extraneous proteins, cell aggregation or glycosylation, may hide cancer antigens or otherwise interfere. Damaged cells, lysed cells, serum, and red cell proteins may also mask cancer cell antigens.

The major histocompatibility complex encodes human leukocyte antigens (HLAs) as part of a system to recognize cells that do not belong in the human body. MHC class 1 HLAs metabolize antigens, then place short derivatives onto cell surfaces to elicit a cytotoxic T-cell response. Vogelstein et al. created antibodies to bind peptide-HLA combinations on cells. (CN108137685A, 2018-06-08). A disadvantage of this approach is that oncogenic mutations persist because of gaps or DNA damage affecting MHC-1 ability to remove abnormal molecules. HLA has more forms than any other human gene, with each version encoding the ability to display a different peptide (Shao et al., 2018). HLA alleles differ widely among cancer patients. Preparing antibodies to abnormal cancer antigens presented in combination with HLAs on cell surfaces ignores data establishing that cancer patients' immune responses are not normal, so effective cancer antigen-HLA combinations are unreliable.

A strategy to produce anti-tumor monoclonal antibodies is to inject whole cells from cancer cell biopsy samples into mice to produce antibodies, generate a humanized hybridoma, and then select a hybridoma antibody cytotoxic to the tumor. (Young, et al., 7,488,475 Feb. 10, 2009; Young, et al., 7,420,039. Sep. 2, 2008; Young, et al., 7,256,272 Aug. 14, 2007; U.S. Pat. No. 7,399,835 Young, et al. Jul. 15, 2008; Young, et al., 7,186,808, Mar. 6, 2007; Young, et al., 6,794,494 Sep. 21, 2004; Young, et al., 7,175,846 Feb. 13, 2007). These prior art methods destroy cancer antigen structure because they require tumor cell fixation in 70% ethanol at −30° C. before injection into mice. The method is long, tedious, and elaborate. Commercial services for hybridoma production require 15-20 weeks. In the interim, the hybridoma protocol selects therapy from a library of premade antibodies. Because cancer heterogeneity extends into billions of different variants, there is little chance that enough different premade monoclonal antibodies will exist. The resulting humanized monoclonal antibody still contains mouse sequences making allergic reactions more likely.

Some cancers, such as melanoma, are treated with immune checkpoint inhibitors. These inhibitors remove self-recognition controls on the immune system and commercial versions warn against subsequent catastrophic organ rejection. Cancer often returns (Anagnostou et al., 2017). Immune stimulating peptides have been patented to treat prostate cancer (Sanda et al., US8 455615), but this strategy also bypasses normal controls on the immune system.

### BRIEF DESCRIPTION OF THE FIGURES

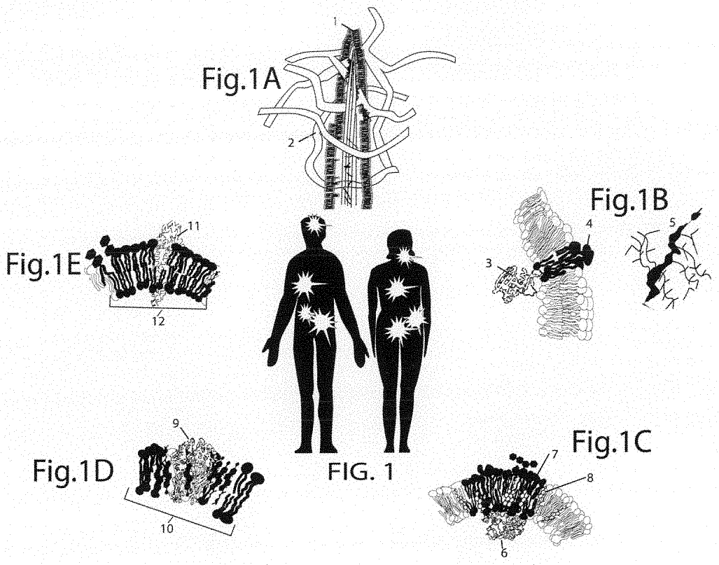

FIG. 1A-E. Abnormal structures on cancer cells provide targets for immunotherapy. Cancer-causing DNA mutations comprise but are not limited to protein structures, membrane structures, lipids, sterols, proteoglycans, phospholipids, sphingolipids, and pathologic cell stromal linkages that are not present in non-malignant cells.

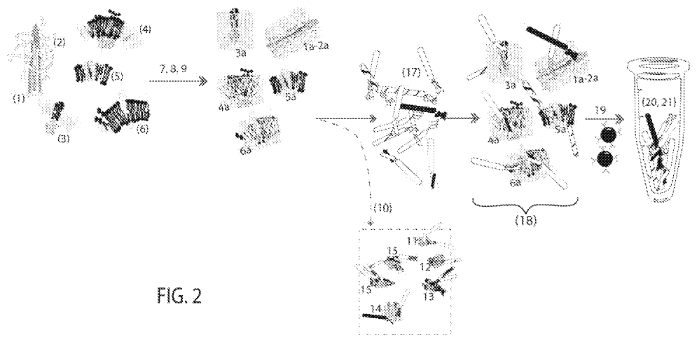

FIG. 2 is a flow chart for the Embodiment 1 protocol to isolate cell surface abnormalities found on cancer cell surfaces but not on normal cell surfaces.

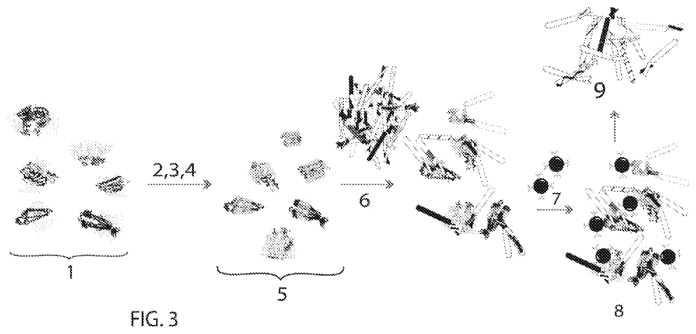

FIG. 3 is a flow chart summary of steps required to edit the phage display antibody library to remove molecules that bind to normal cells. The figure illustrates examples of membrane proteins, but the process also comprises normal counterparts of cancer cell structures affecting lipids, sterols, proteoglycans, phospholipids, sphingolipids, and stromal linkages.

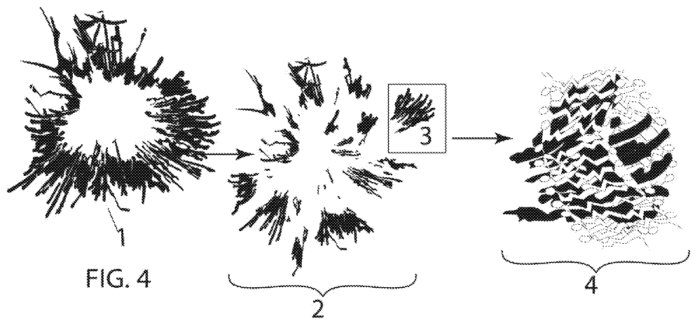

FIG. 4 illustrates an amphipathic agent solubilizing a highly abnormal cancer cell membrane to retain surface structures in aqueous solutions.

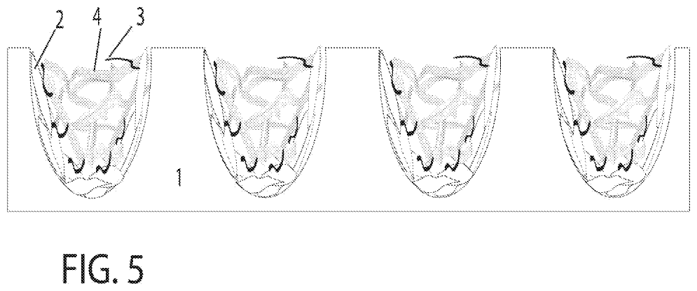

FIG. 5 represents an artificial mimic of the extracellular medium designed to preserve the shapes of whole cancer and normal cells.

### OBJECTIVES OF THE INVENTION

AIM 1 of the embodiments is to provide antibody-like molecules that can be used to treat virtually any cancer, without damaging non-malignant cells

AIM 2 of the embodiments is to provide antibody-like reagents which can serve as a pharmaceutical platform for derivatives which could augment therapeutic effectiveness in the treatment of cancer.

AIM 3 of the embodiments is to improve available therapy for cancer, regardless of its stage, grade.

AIM 4 of the embodiments is to provide therapy when standard therapy has either failed or become too toxic to continue.

AIM 5 of the embodiments is to produce specific anticancer antibody-like molecules quickly enough for them to become an option for primary therapy.

These and other aims of the invention are clear from the description herein.

### BRIEF DESCRIPTION OF THE INVENTION

The embodiments overcome the disadvantages of the prior art and use the discovery of cancer's abnormal cell morphology to guide methods to prepare therapeutic reagents. The procedures contain sufficient detail so that the skilled practitioner can use them for any type of cancer at any stage or grade. Cancer-specific cell structural aberrations are universal in cancer and distinct from prior art involving neoantigens or other cancer antigens. A significant aspect of the embodiments preserves cancer cell surface morphology as a new process derived from methods to determine three-dimensional protein structures. Systems and methods select and amplify therapeutic antibody-like molecules specific for cancer cell surface structures that do not exist on normal cells.

The embodiments supplement the universal immune deficit in cancer to compensate for it. Polyclonal immune supplements make the embodiments independent of the number of different malignant species present and the development of new malignant clones. The design solves the problem of rare surface antigens such as those on cancer stem cells by amplifying binding molecules to any desired level. The embodiments remove antibody fragments that react with normal cells, preventing normal cells from being targeted. The embodiments increase the concentration of molecules specific to cancer cell surfaces to improve the ability to select cancer binding fragments from large phage display libraries. The embodiments are superior to targeted therapy because they do not require identifying shared mutated genes or even which type of cell is malignant. Unlike checkpoint inhibitors, the immune supplements do not remove essential controls on the immune system that permit self-recognition. A skilled laboratory can produce cancer binding antibody fragments fast enough to add polyclonal immunotherapy reagents to primary cancer therapy.

Subtracting out antibodies that bind normal off-target organs increases safety. Because of the increase in safety, the embodiments provide a bridge preventing tumor regrowth during periods when organ toxicity forces patients to discontinue or stop conventional therapy. Another aspect of the embodiments are their speed of implementation. Another aspect is preserving the morphology of cells and cell surface molecules, whether they represent cancer- or non-malignant cells. Other aspects are new uses for rapid methods to enrich cancer cell-specific molecules from the surfaces of cancer cells and to assay cancer and normal cell surface molecules for the preservation of their 3-dimensional structures.

### Significant Aspects of Invention Embodiments

A major aspect of these embodiments is that they preserve the 3-dimensional structures of molecules specific to cancer cell surfaces and absent from non-malignant cells.

A significant aspect of the embodiments is the prohibition of chemical cross-linking fixatives, organic solvent precipitation, coagulation, physical fixative reagents, or any other conditions that change the 3-dimensional structure of cancer cell surface antigens.

Another aspect of the embodiments is that therapy with antibody-like molecules does not identify or target cancer cell driver mutations.

Another aspect of the embodiments is that they do not depend on the cell, tissue, or organ from which cancer originates.

Another aspect of the embodiments is that they do not depend on stratifying patients based on the presence or absence of specific driver gene mutations or prognostic factors.

Another aspect if the embodiments is that they comprise rapid production of antitumor binding molecules so that they said molecules can contribute to primary anticancer therapy

### DETAILED DESCRIPTION OF THE INVENTION

Abnormal surface morphology in cancer has never been recognized either as a general basis for therapy or a general distinguishing characteristic. Preserving the structure and arrangements of abnormalities on cancer cell surfaces are essential to produce therapeutic antibody-like molecules. With reference to FIGS. 1-4:

The aggregate total of DNA mutations, breaks, deletions, insertions, and rearrangements in heterogeneous populations of cancer cells with diverse genetic mutations create abnormal cancer cell surface structures. FIGS. 1A-E illustrate cancer-induced changes in proteins, in the asymmetric distribution of phospholipids, and in other molecules in the inner and outer cell membranes of lipid bilayers. These changes comprise all sites susceptible to cancer, some of which are indicated on the male and female drawings. Mutations responsible for these abnormalities create myriad targets for immunotherapy. Membrane proteins, phospholipids, sphingolipids, proteoglycans, and sterols depend on countless steps and interactions in metabolic pathways, all subject to mutation. Cancer DNA further causes abnormal cell morphology because mutations affecting some membrane proteins and sterols reorganize, harden, or soften membranes. Qui et al. (2020) demonstrated that side chains of membrane proteins cause marked ordering of the membrane around integral proteins. Mutations disrupt this ordering creating potential immunotherapy targets. FIG. 1 comprise examples of typical cancer cell structures with information on how they embed in the membranes. The sample proteins are individually labeled based on silhouettes obtained from the Protein Data Base (PDB). The cell membrane is a lipid bilayer that displays cancer cell associated structures. The lipid bilayer is composed of an inner and outer layer which differ in composition. A small animal cell contains about 1 billion molecules in its cell membrane, with phospholipids being the most abundant. The bilayer also includes sphingolipids and interspersed sterols such as cholesterol (depicted), which generally stiffens the bilayer. Typically, 1300-1500 different phospholipid species distribute differently between the inner vs. the outer plasma membrane and help determine cell shape (Llado et al., 2014.) In FIG. 1A-E, cancer cell DNA disrupts a myriad of normal interactions with diverse molecules. This disruption creates pathologic structures that are not present in normal cells.

FIG. 1A depicts an invadopodium (1) from a cancer cell surface that breeches extracellular membrane structures (2) and begins cancer spread from its source cell. Invadopodia are known to unmask and distort proteins and release proteolytic enzymes, facilitating invasion and metastasis (Hoshino et al. J. Cell Science 2013). Invadopodia are likely in breast, colorectal, bladder, and head and neck squamous cell carcinoma (Yamaguchi 2012).

FIG. 1B represents the CD44 cell surface molecule (3) that becomes mostly incompatible with its normal display on the cell surface because of a DNA mutation. Absence of CD44 creates abnormal membrane structures (4) (black). The cancer cell lacking surface CD44 can no longer bind extracellular hyaluronic acid (5) so the cell loses a positional anchor. CD44 variations have been linked to non-Hodgkins lymphomas, colorectal cancer and breast cancer.

In FIG. 1C, a Src enzyme (6) inside the cell (cytoplasmic) is an enzyme associated with the cell membrane. Here, Src mutates and alters membrane structure (shown in black). The Src mutation changes the cancer cell membrane by altering the distributions of membrane constituents such as phospholipids and sphingolipids (7) and cholesterol (8). (indicated in black). Prostate, lung, breast and colorectal cancers involve Src.

FIG. 1D. Integrins are transmembrane receptors that mediate cell adhesion. Inactive integrin does not associate with lipid rafts but activated integrin moves laterally using lipid rafts. Cytoplasmic tethering restrains integrin from boarding the rafts. (Leitinger and Hogg, 2001).

FIG. 1D shows integrin mutation creating distinctive cancer immunotherapy targets because it alters not only integrin (9) but also the cancer cell membrane (indicated by black structures). Integrin mutation and a cholesterol-sphingolipid enriched raft (10) have ferried mutated integrin (9) laterally to a new location, altering cell shape and adhesion reactions with other cells. Melanoma, glioblastoma, and cancers of the breast, prostate, pancreas, ovary, lung, colon, and cervix have abnormal integrins.

In FIG. 1E, Tetraspannin ordinarily regulates transporting molecules into structurally distinct, small, specialized cell membrane regions. A non-functional Tetraspannin protein (11) causes changes in distributions of (black) membrane components (12), thereby altering cell morphology. Tetraspannins participate in chronic lymphocytic leukemia, lymphoma, melanoma and cancers of the breast, esophagus, kidney, bladder, and lung (non-small cell),

### Embodiment 1 (FIG. 2)

Embodiment 1 takes advantage of cancer cell surface deformations caused by mutations and their effects to alter membrane-proteins. Embodiment 1 selects and amplifies cancer binding molecules for rapid, routine cancer immunotherapy. The most critical factor in using these cell surface formations as immunotherapy targets is to preserve their 3-dimensional structures and arrangements. If cancer cell surface structures become artificially distorted, protocols will select antibodies that recognize the distorted cancer cell surfaces. These antibodies have a high affinity for the distorted antigen but not for the antigen in tumor samples.
1. Cancer cells comprise structures (1-6) on the same or different cell populations and contributions from the microenvironment (2) such as those from FIG. 1
7. Preserving cancer cell structures begins by enriching for the outer cell membrane surfaces. The Membrane Fraction preparation comprises gentle lysis and differential centrifugation at speeds routinely used to prepare cell membrane fractions (Suski et al., 2014), e.g., 25,000 g and 100,000 g. Preservation of abnormal structures such as those comprising (1-6) is monitored by full or partial retention of activity to a panel of fluorescent antibodies known to react with cell surface molecules such as CD24, CD44, CD142, CD144, CD146, CD171, CD220, CD221, ERBB1, SSEA-1.
8. Combinations of one or more “Structure-Preserving Detergents” disperse the membrane fraction (7). Because of differences among cancers in different organs, it is unlikely that any single detergent will always be the best choice. Five of the most widely used detergents work at concentrations of 1-2%: Dodecyl mannoside, nonyl glucopyranose, octyl glucopyranoside, lauryl maltose neopentyl glycol, and lauryl dimethyl amine N-oxide. X-ray crystallography and other structural studies validate the ability of these detergents to preserve protein three-dimensional structures. (Mus-Veteau I. 2014; Bonnete and Loll, 2017; Stetsenko, 2017). Again, reactivity to a panel of known anti-cell surface antibodies monitors the preservation of three-dimensional structures by the detergents selected for each individual cancer. The binding of fluorescent derivatives of anti-cell surface antibodies is measured by flow cytometry.
9. The addition of amphipathic stabilizing agents such as biotinylated amphipol A8-35 at about three times the weight of membrane structures stabilizes abnormal versions of surface structures (comprising structures labeled as 1a-2a, 3a, 4a, 5a, and 6a) to safely remove detergent (step 7). Trapping in the amphipathic agent requires adjusting NaCl concentration to 100 mM and detergent to near its critical micellar concentration (0.2 mM for dodecyl maltoside). Optimizing stabilization requires determining the amount of amphipol to maintain abnormal structures in an aqueous solution after removing detergent. Adsorption onto polystyrene beads (Bio-Beads at 4° C. for 3 hrs.) or simple dilution prevents detergent interference, which usually does not cause problems (Zoonens et al., 2014).
10. Editing a phage display library subtracts antibody-like molecules that bind non-malignant cells (10). Modern phage display technology allows reliable access to extensive antibody libraries. These libraries yield anti-cancer cell surface protein antibodies that permit enormous amplification without lengthy immunizations, without degrading cancer cells, and without changing their cell morphology.
Targeting cancer cell surfaces lowers the number of antibodies required since cell surfaces contain only a small fraction (about 6000) of total cellular proteins. The phage (bacterial virus) genome includes most human antibody binding region genes within its DNA as placed by recombinant DNA technology. When the phage infects bacteria such as e coli, the phage uses bacterial machinery to make (“display”) the binding regions of human antibodies as part of its outer coat. Phage display technology also facilitates removing off-target antibodies (enclosed by dashed line in box),
The phage display library initially comprises 1-100 billion different species, comprising antibody-like fragments that react with normal cells in addition to those that react with malignant cells. Normal cell surface structures trapped in amphipathic agent react with the phage display antibody library to edit it by removing molecules that bind normal cells (11-16), minimizing toxicity to normal cells. Normal examples comprise normal Src kinase membrane complex (11), CLCA1 (12), Multidrug transporter ABCB1 (13), normal integrin (14), normal Tetraspannin CD81(15), and carbonic anhydrase. Because the molecules shown binding structures 11-16 are single chain (ScFv) derivatives, they contain only one binding site and will not cause cross-linking and precipitation in the stabilized environment. The presence of biotin on the amphipathic stabilizing agent enables easy removal of these anti-normal cell molecules.
17. The edited phage display library (17) recognizes amphipol-trapped cell surface proteins and other molecules, labeled as 1a-6a. After binding to the amphipathic stabilizing agent, the detergent is removed by adsorption to polystyrene beads (Bio-Beads)
18. Members of the edited phage display library bind to abnormal cancer cell surfaces (1a-6a). It is not necessary to know the identity of the surface molecule or the cell of origin. All that is required is that the surface molecule does not belong within any non-malignant cell environment.
19. Cancer cell surfaces as biotin-amphipathic derivatives bind to a streptavidin containing solid immunoabsorbent (as in FIG. 3). Phages displaying irrelevant antibody fragments do not stick and wash away. Biotin and streptavidin bind very rapidly, forming the most stable known non-covalent bond between a protein and a ligand. Broad extremes of pH, temperature, organic solvents, and other denaturing agents do not disrupt the complex. The tenacious biotin-streptavidin bond prevents excess amphipol from interfering with subsequent elution of the cancer cell binding molecules. Low pH or trypsin then elutes only the bound antibody phages, which can then reinfect bacteria (usually e. coli).
20. The number of e coli cells doubles every 20 minutes, and infected cells multiply faster. Moreover, an infected cell produces 1000 progeny bacterial viruses in one hour (Smeal et al., 2017). Bound antibody phages are eluted, used to reinfect more e. coli to make more specific antibody phages.
21. Repeating these steps three or more times yields large amounts of phage-displayed antibody mixtures at billion-fold levels of amplification. The capacity for virtually unlimited amplification makes it feasible to produce therapeutic antibodies to very rare cancer-specific antigens. The speed of phage display antibody production permits using it for truly personalized individual therapy. Diversification in error-prone e. coli further increases the potential number of antibody-like molecules.

FIG. 3. Solid-phase immunoadsorption subtracts antibodies to non-malignant cell surfaces and the amphipathic reagent such as biotinylated amphipol A8-35. The phage-display library initially contains 1-100 billion different binding molecules.
1. Normal cells from tumor margins clear of malignancy, any additional available non-malignant normal tissue, and non-malignant finite cell lines undergo the same procedures as the tumor cells (FIG. 2). Removing phage display antibodies to non-malignant cell surfaces produces the “edited” antibody phage display library used in FIG. 2. This subtraction enriches the library in antibody-like molecules that bind cancer cell surfaces.
2. The normal cell samples undergo gentle lysis and differential centrifugations to prepare membrane fractions.
3. The same detergents used for cancer cells (FIG. 2) disperse the cell surface structures.
4. Biotinylated amphipol then stabilizes non-malignant cell surface structures as shown, substituting for the original cell membrane. The detergent can then be removed by adsorption to polystyrene (Bio-Beads).
5. Antibody fragments from the phage display library (6) bind the amphipol stabilized structures (5) react with antibody derivatives within the phage display library (6), comprising 1 to 100 billion molecular species. Binding is measured by flow cytometry of fluorescent antibody derivatives.
6. The phage antibody derivatives (ScFv fragments) in the library comprise 1 to 100 billion binding regions, which differ at the molecular level. Each member of the library has only a single binding site, so it cannot cause cross-linking or precipitation.
7. Solid streptavidin particles (7) then bind biotin groups on the amphipathic stabilizing agent.
8. The binding of streptavidin beads immobilizes biotinylated amphipol derivatives of surface molecules from normal cells. Phages that display antibodies to normal cell surface molecules will stick, allowing their removal. The phages that do not stick contain binding molecules specific to cancer cell surfaces and irrelevant antibody fragments.
9. An “edited” phage display library then will not react with surface proteins from the normal cells.

FIG. 4. The membrane structure in a cancer cell is no longer regular. Invasive membrane structures that differ from non-malignant cells include invadopodia (invasive actin-rich projections), blebs (membrane bulges), and membrane ruffles. At least one antibody can recognize invadopodia in cancer. (Baik et al., 2019).

Mutations accumulate and may generate highly abnormal cancer cell membrane structures (10 and grossly malformed cancer cells. Inappropriate mixing of the inner and outer cell membrane components, mutations in biosynthetic pathways, and changes in protein structure produce aberrant cell membranes. The illustration shows an example of an abnormal membrane fragment patterned after known cancer cellular abnormalities. When the protocol in FIG. 2 is applied, pathologic phospholipid and other membrane assemblies (black) disperse on cell lysis and structure-preserving detergents (2). Amphipathic agents such as biotinylated amphipol A8-35 stabilize the abnormal structures. For example, one part of the abnormal structure (3) produces the stabilized aqueous compatible derivative (4). Structure 4 and the remaining other parts of the cancer cell surface select binding antibodies from an edited phage display library as in FIGS. 2 and 3.

### Embodiment 2 (FIG. 5)

To preserve the structure of normal cell and cancer cell antigens, cells are immobilized in microwells on a microplate (“1” in FIG. 5) under conditions that maintain in vivo morphology. Before exposure to a patient derived cancer cell specimen, we generate conditions mimicking cancer cell physiologic environments. First, microwell plates are coated with stromal cells (“2” in FIG. 5.) (such as fibroblasts or smooth muscle cells). Next comes a combination of fibronectin and hyaluron added in sequence (“3” in FIG. 5). Human fibronectin (0.1%) facilitates adhesion through binding to integrin receptors (1-5 mcg/cm2). Hyaluronic acid coating is done by the method of Corradetti et al., 2017. The last addition is animal origin-free recombinant Type 1 human collagen (“4” in FIG. 5) in dilution medium to encourage adhesion.

After cancer cells are placed on the substrate, the cells are exposed to an edited phage display library. The library has been edited so that it does not bind the substrate or normal cells in the cancer cell preparation.

Embodiment 2 works if the cancer cell membrane structure is highly abnormal, as in FIG. 1D or of the cancer cell surfaces are fragile. Lysis, binding, detergent, and amphipathic agent suspension are then conducted as in FIG. 2. A competitive ELISA assay tests for distortion after cancer cells are immobilized.

Embodiments 1 and 2 remove phage display antibody fragments that bind to normal cells under the same conditions used to prepare the immunoabsorbent for cancer cells. The preservation of normal cell morphology is determined by maintaining reactivity to one or more members of a panel of antibodies directed against non-malignant cell surfaces.

Cancer cells are then exposed to the antibodies that do not bind in either embodiment 1 or 2. Washing removes irrelevant antibodies so antibodies specific for cancer cells can be recovered, amplified, and purified.

The phage particles that carry the cancer-specific antibodies have an engineered trypsin cleavage site making it convenient to release bound phage particles. Limited trypsin digestion releases bound phages that are still infectious and still produce antibody-like fragments such as ScFv, Fab, FHH, or other forms. The released phage particles then reinfect e-coli. A few cycles of reinfection and re-release yield highly purified anti-cancer cell therapeutics with billion-fold amplification.

Diversify the anti-cancer antibodies. One advantage of the anti-cancer antibody-like fragments is that they are mixtures (polyclonal), capable of forming large, tight complexes. Another aspect of these mixtures includes recognizing different parts of the same cancer, reacting with varying antigens of cancer in other parts of the same tumor, and in tumor metastases. Antibodies are amplified in error-prone e-coli to diversify the anti-cancer antibodies, increase binding avidity, and produce binding to rare cancer stem cell antigens. Even with the time it takes for affinity maturation and to add effector functions, it is much faster to produce patient-specific anti-cancer cell surface binding antibodies than to produce hybridoma antibodies.

Production of Cytotoxic Antibodies.

ScFv fragments are converted to ScFv-Fc fragments according to the prior art. For example, TGEX-SCblue is a commercially available mammalian expression vector designed to convert ScFv phage clones into bivalent ScFv-Fc fusion proteins that can be quickly expressed in HEK 293 cells (Jager et al., 2013). The conversion begins with phagemids with antibody V genes inserted between two SFiI sites. The V-genes are then cut and pasted into a mammalian expression vector by restriction fragment cloning without needing PCR amplification (Yoon et al., 2012). Typical yields are at least 10-100 mg/ml in a few days. The product reproduces the binding of a fully reconstructed monoclonal antibody. Transient transfections are easy in 24- and 48-well plates in parallel.

The prior art also describes preparing other cytotoxic antibody-like derivatives such as diabodies, Triomab, Fab2, Tandem ScFv, and multi-specific forms.

Radioactive derivatives or combinations with cytotoxic drugs are also readily prepared according to prior art.

The immunotherapy anticancer binding molecules may be coadministered with standard cancer treatment.

The invention claimed is:

1. A method of preparing membrane fragments from heterogeneous cancer cell mixtures from an individual patient comprising:
a. altered cancer cell morphology caused by the cumulative results of all the DNA mutations, deletions, insertions, and rearrangements in the patient's cancer cells;
b. abnormal cell surface molecular arrangements, comprising phospholipids, sphingolipids, lipids, sterols, proteoglycans, carbohydrates, and proteins in said cancer cells;
c. the preservation and maintenance of the three-dimensional arrangements of molecules on cancer cell surfaces from an individual patient comprising those that differ from normal cells matched to the same patient;
d. said preservation and maintenance is achieved without the use of fixatives, organic solvents, formaldehyde, glutaraldehyde, other cross-linking agents, heat, or other methods of coagulation.

2. Methods comprise producing cancer immunotherapy medication by using said unnatural surface morphology created by cancer DNA modifications, characteristic of an individual cancer, and absent from non-malignant cells matched to the individual patient.

3. Methods comprise preparing and isolating polyclonal anti-cancer binding molecules specific to the abnormal morphology of cancer cells in the individual, regardless of the rarity of the feature.

4. Methods comprise preparing immunotherapy reagents and immunodiagnostics for distinguishing cancer vs. normal cells in any individual patient;
a. Methods comprise the selection and purification of polyclonal immunotherapy agents that are unlikely to react with tissues that do not contain malignancy.

5. Methods are prompt and rapid enough to prepare cancer immunotherapy reagents specific for cancer in one individual patient.
